(19) United States
(12) Patent Application Publication
(10) Pub. No.: US 2015/0209561 A1
(43) Pub. Date: Jul. 30, 2015

(54) Wearable Topical Drug Delivery Device

(71) Applicant: Edward Wesley Everhart, Farmington, UT (US)

(72) Inventor: Edward Wesley Everhart, Farmington, UT (US)

(21) Appl. No.: 14/313,381

(22) Filed: Jun. 24, 2014

Related U.S. Application Data
(60) Provisional application No. 61/932,284, filed on Jan. 28, 2014.

(51) Int. Cl. A61M 35/00; A61K 9/70

(52) U.S. Cl. CPC A61M 35/00; A61K 9/703

(57) ABSTRACT

The present invention provides a wearable drug delivery device that is adapted to be worn by a user. The device can be provided as strips of a porous mesh material for wrapping around the body or as wearable garments that are impregnated with natural medications and stimulants. The materials may be wrapped around the desired portion of the body such as the forehead, chest, hands, wrists, knees, or feet in a variety of ways. The material may be impregnated with a multitude of natural medications and stimulants including iodine, caffeine, hormones, pain medication, diet drugs, argentine, taurine, vitamin B, vitamin D, ginseng, and green tea, among others. The present invention enables the localized application of topical medications, thereby providing direct treatment to the area of the body in need.

### CROSS REFERENCE TO RELATED APPLICATION

This application claims the benefit of U.S. Provisional Application No. 61/932,284 filed on Jan. 28, 2014. The above identified patent application is herein incorporated by reference in its entirety to provide continuity of disclosure.

### BACKGROUND OF THE INVENTION

1. Field of the Invention

The present invention relates to a topical drug delivery system. More specifically the present invention relates to one or more impregnated bands that can be wrapped around the body and worn under the clothing of an individual in order to deliver nutrients directly to the points of need of the body of an individual.

Medications can be delivered in a multitude of ways into the body of an individual. They can be inhaled, injected, ingested, and absorbed through the skin, and each is suitable for their own needs. Of medication delivery methods, application of the medication through the skin is the method most suitable for localized application, whereas inhalation, injection, and ingestion introduce the medication throughout the entirety of the body. Many medications have to be directly applied to the surface of the body of individuals in order to provide a localized treatment on the body. Creams, gels, foams, lotions, and ointments can be applied topically onto the body to treat rashes, acne, burns, pains, and the like.

While topical medications apply the medication directly to the source of treatment, drawbacks exist to the traditional application of the medication. Many topical drugs are utilized for treating skin conditions, such as skin inflammations, burns, and other dermatological conditions. While suitable for treating the skin condition, the problems often persist because the skin may become irritated by the clothing worn over the body, and because of this individuals may opt to avoid treatment. Additionally, many topical treatments require frequent applications of medication or may call for spaced apart treatments, both of which require an individual to actively remember to apply the medication. Still further, depending on the treatment site, an individual may not be capable of reaching the location to apply the medication without assistance.

People often develop solutions for overcoming the drawbacks remembering when to take medication, which applies to inhaled, injected, ingested, and topically applied medications. Common solutions involve taking medication at recurring times through-out the day, such as when eating, when waking up, going to sleep, or by setting an alarm. These solutions, however, require an individual to carry their medication with them and assume that the medication can be easily taken when away from the home, and therefore can be easily forgotten or neglected. Moreover, in the case of topical medications, there can be increased obstacles to applying the medication. Clothing may have to be removed prior to application of the medication, and depending on the skin condition, the friction between the clothing and the skin may increase the problem that needs to be treated.

There are several prior art devices that provide transdermal drug delivery systems. Some of these devices provide a layer incorporated with a drug that is released transdermally into an individual, whereby the transferred solution can include minerals, nutritional supplements, drugs, vitamins, and the like. These prior art devices, however, fail to provide bands or strips of material impregnated with a treatment solution therein for transdermal application into the skin, and further fail to provide body garments adapted for use under clothing.

The present invention, however, provides a wearable drug delivery system that is impregnated with natural medications and stimulants. The materials may secured around the body by wrapping or can be provided in the form of clothing, whereby the device can be worn around any portion of the body such as the forehead, chest, hands, wrists, knees, or feet in a variety of ways. The material may be impregnated with a multitude of natural medications and stimulants including iodine, caffeine, hormones, pain medication, diet drugs, argentine, taurine, vitamin B, vitamin D, ginseng, and green tea, among others in order to provide a localized medication treatment that is absorbed into the problem area of the body.

2. Description of the Prior Art

Devices have been disclosed in the prior art that relate to topical drug delivery systems. These include devices that have been patented and published in patent application publications. These devices generally relate to patches for actively applying medication onto the body. The following is a list of devices deemed most relevant to the present disclosure, which are herein described for the purposes of highlighting and differentiating the unique aspects of the present invention, and further highlighting the drawbacks existing in the prior art.

One such prior art device, U.S. Patent Publication No. 2008/0233180 to Zeltman provides a transdermal patch with a solution of vitamins thereon, such as vitamin B12. The patch can include a skin adhesive layer and an occlusive layer for preventing the loss of medication prior to use. Similarly, U.S. Patent Publication No. 2004/0208916 to Abbot provides a transdermal patch with an adhesive and an impermeable layer to prevent a loss of therapeutic substances prior to use. The prior art devices, however, fail to provide strips of material for wrapping around a portion of an individual's body, and further fails to provide garments impregnated with a topical solution as is provided by the present invention.

U.S. Pat. No. 6,461,644 to Jackson provides a topical drug delivery patch with a reservoir layer having a thickness that provides for the sustained administration of nutrients to the body of an individual. The prior art device, however, fails to provide a garment shaped topical drug carrier for providing the continued release of a topical drug to an individual.

Yet another device, U.S. Patent Publication No. 2012/0220962 to Hsu provides a method for enhancing the permeability of topical or transdermal medications. Although the prior art method provides a means for applying medication to the body, the prior art method fails to provide a garment for wearing on portions of the body.

Finally, U.S. Pat. No. 5,028,435 to Katz provides a system and method for transdermal drug delivery, whereby the system comprises a matrix layer impregnated with a drug and further includes a drug release enhancer for speeding the process of delivering medication to the body of an individual. While similar in nature and relevant to the present invention, the prior art system and method fail to provide a garment shaped topical drug delivery device.

The present invention, however, provides a wearable device that comprises a woven mesh material having pores thereon, whereby the pores are impregnated with a topical medical solution thereon. The device can be provided as an elongated strip of material, similar to a medical wrap, or as a garment that can be worn over a portion of the body of an individual. The device can be worn or wrapped on or around the forehead, chest, hands, wrists, knees, or feet in a variety of ways. The material may be impregnated with a multitude of natural medications and stimulants in order to facilitate the localized application of topical medications, thereby providing direct treatment to the area of the body in need.

It is submitted that the present invention is substantially divergent in design elements from the prior art and consequently it is clear that there is a need in the art for an improvement to topical drug delivery devices. In this regard the instant invention substantially fulfills these needs.

### SUMMARY OF THE INVENTION

In view of the foregoing disadvantages inherent in the known types of topical drug delivery devices now present in the prior art, the present invention provides a new topical applicator wherein the same can be utilized for providing convenience for the user when desiring the application of a topical drug on the body for an extended amount of time.

It is therefore an object of the present invention to provide a new and improved topical drug delivery device that has all of the advantages of the prior art and none of the disadvantages.

It is another object of the present invention to provide a wearable topical drug delivery device.

Another object of the present invention is to provide topical drug delivery device that can be wrapped around the body in order to provide treatment thereon.

Yet another object of the present invention is to provide a container for housing a topical drug delivery device and a medical solution therein.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 displays a view of the drug delivery device of the present invention and a device storage container.

FIG. 2A displays a view of the present invention wrapped around the upper torso of an individual.

FIG. 2B displays a reversed view of the present invention wrapped around the upper torso of an individual.

FIG. 3A displays a view of the present invention wrapped around the hand of an individual.

FIG. 3B displays a reversed view of the present invention wrapped around the hand of an individual.

FIG. 4 displays an alternative embodiment of the present invention, whereby the drug delivery device is designed as wearable garments.

### DETAILED DESCRIPTION OF THE INVENTION

Reference is made herein to the attached drawings. Like reference numerals are used throughout the drawings to depict like or similar elements of the drug delivery device of the present invention. For the purposes of presenting a brief and clear description of the present invention, the preferred embodiment will be discussed as used for providing a means for transferring a topical medication onto the body of an individual. The figures are intended for representative purposes only and should not be considered to be limiting in any respect.

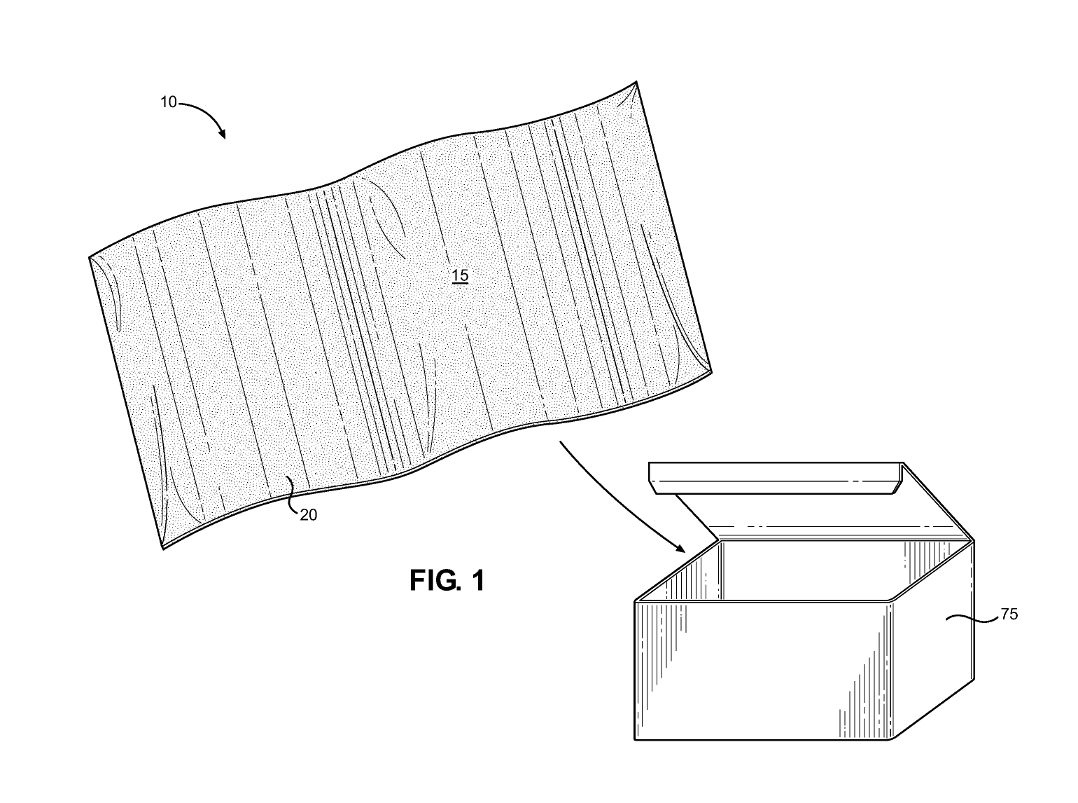

Referring now to FIG. 1, there is shown a view of the drug delivery device of the present invention and a device storage container. The material 10 provides a device for delivering a topical medication onto the body of an individual. The device is constructed of a continuous piece of porous material 15 such as a woven mesh, compression fabric, cloth, or baby wipe. The pores 15 of the device 10 are impregnated with a topical medical solution for the treatment of a variety of skin conditions, such as skin inflammations, burns, acne, and other dermatological conditions. The pores 15 can be impregnated with one or more treatment solutions 20, including but not limited to: iodine, caffeine, hormones, pain medication, diet drugs, argentine, taurine, vitamin B, vitamin D, ginseng, green tea. Additionally, the device 10 can be provided with a mixed solution that is specifically designed for the treatment of a singular condition. For example, for acne treatment, the solution can comprise one or more of benzyl peroxide, tea tree oil, salicylic acid, lavender, eucalyptus, lemon juice, or another acne-fighting solution.

The drug delivery device 10 may be provided within a sealed package 75. The package can be offered in a sealable bag or hard case with a screw-on lid to remain moist before use. Alternately, the material can also be packaged in a dry bag or case, thereby allowing people to apply a measured amount of a medical solution onto the drug delivery device prior to application onto the skin. In this way, the present invention provides individuals with an easy and simple way to apply acne treatment directly to their bodies.

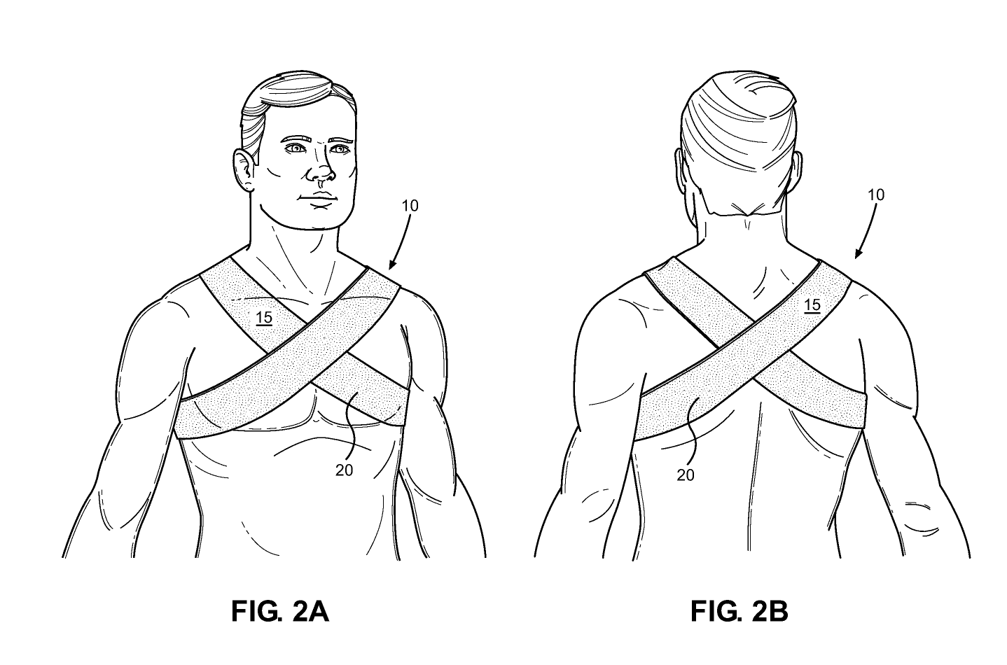

Referring now to FIGS. 2A and 2B, there are displayed front and reversed views of the drug delivery device of the present invention wrapped around the upper torso of an individual. The drug delivery device 10 can be offered as an elongate sheet of material that can be wrapped around the body in a variety of ways in order to suit the needs of an individual. For example, the drug delivery device can be utilized similar to a medical wrap. The device can be wrapped around the feet, knees, legs, arms, elbows, chest, wrists, and the like in order to provide for the targeted delivery of a medical solution 20 through the pores 15 thereon.

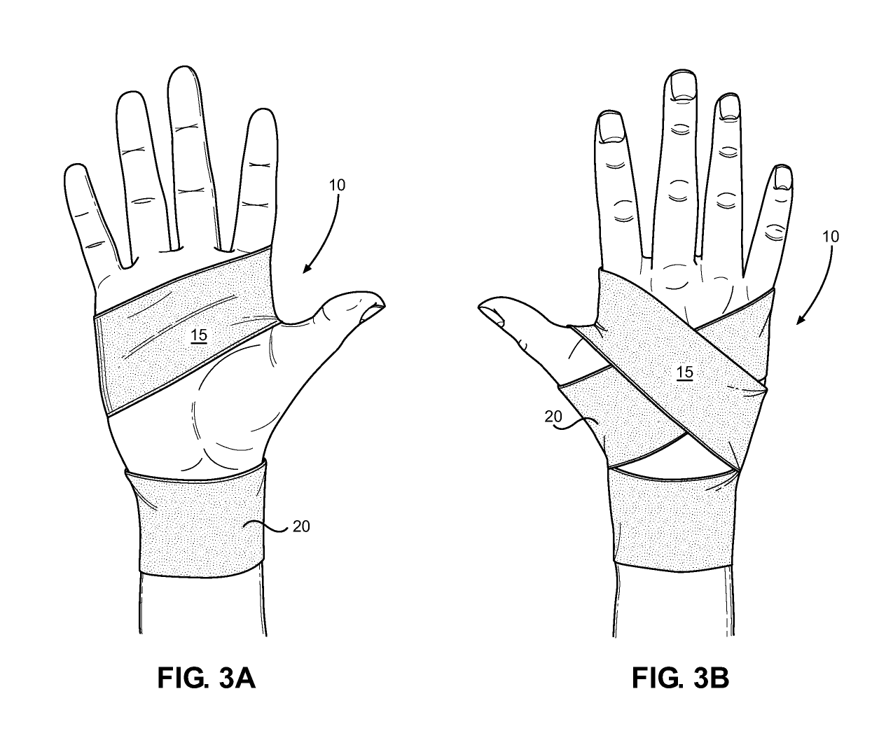

Referring now to FIGS. 3A and 3B, there are displayed views front and reversed views of the drug delivery device of the present invention wrapped around the hand of an individual. The present invention can be offered in a variety of lengths, or alternatively, the elongate sheet provided by the present invention can be cut into different lengths in order to provide for fitting on different parts of the body, such as the hands and wrists. After wrapping, the present invention can be tucked underneath a portion of the material, tied, or clipped onto an opposing end of the sheet in order to provide a secure and tight fitting against the body of the individual.

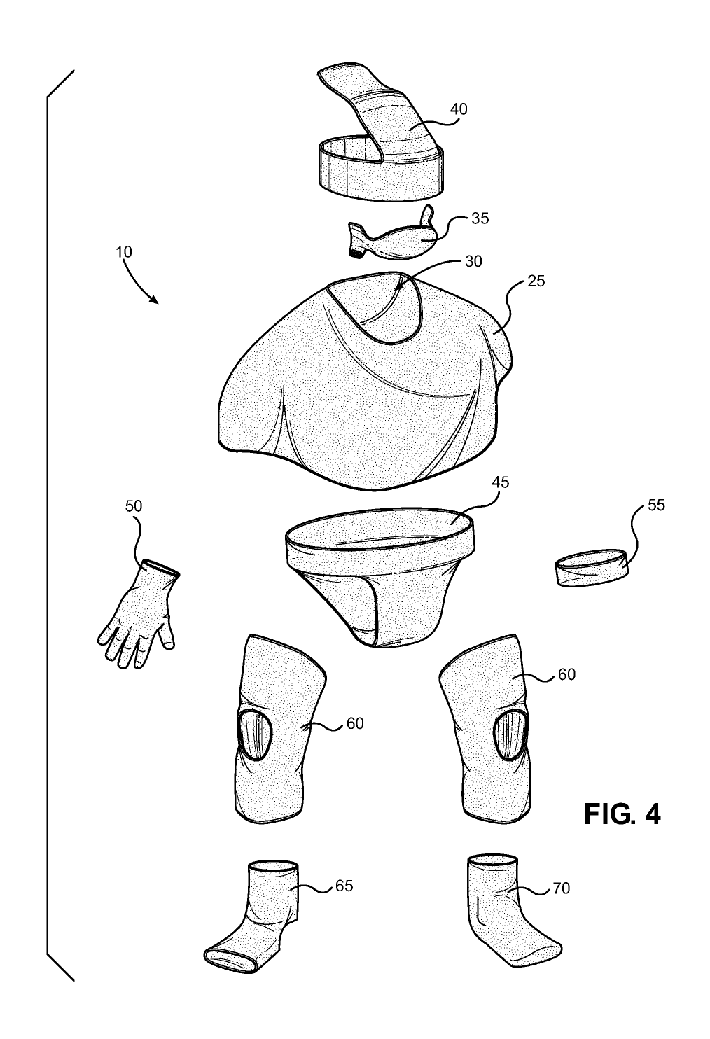

Referring now to FIG. 4, there is displayed an alternative embodiment of the present invention, whereby the drug delivery device 10 is designed as wearable garments. The topical medication delivery device can be utilized as a one-time applicator or as a reusable means for topically applying a medical solution onto the body of an individual. Additionally, the device can be provided in the form of garments which can be designed for placement on different areas of the body such as the head, shoulders, chest, back, legs, arms, hands, feet, chin, and the like.

In one embodiment, the present invention can be offered in the form of a chest and shoulder covering 25 having a sides and a central opening 30 on an upper surface thereof that is sized for enabling an individual to receive their head therethrough, whereby the garment 25 rests over the upper chest, back, and shoulders of an individual in order to provide treatment thereon. The device 10 is placed directly onto the skin and is sized to fit underneath clothing that covers the upper torso of an individual. The chest and shoulder covering 25 can be worn under shirts or underneath sports equipment, such as shoulder pads.

The present invention can also be offered for providing treatment onto the head and forehead areas of an individual. The present invention 10 of the alternative embodiment comprises a head wrap 40 adapted to be placed about the circumference of the user's head, providing coverage for the user's forehead. The device can also be provided with an upstanding flange for placement onto the scalp of the user.

To provide treatment to the scalp and forehead of an individual, the present invention can resemble a skullcap, headband, neckerchief, or tube-style cap. The medication delivery device of the embodiment can be modified for a fit within sports helmets and align with the padding thereof such that skin pressure points are provide coverage by the material and no exposed skin makes direct contact with the athletic gear. Additionally, the device can be offered for fitting beneath hats or can be provided for use without a protective accessory such as hats or sports gear. To provide a more secure fitting, the head treatment wrap 40 may comprise a securing connector element or a continuous, elastic structure to prevent the material from becoming displaced during strenuous activities.

The drug delivery device 10 can further be provided for treatment on the chin of an individual. The drug delivery device can be offered as a cup-shaped insert 35 that is designed to be fit into a protective chin guard required for contact sports that require such protection. Additionally, the drug delivery device can be offered in a variety of other garment types, including that of gloves 50, undergarments 45, wrist/armbands 55, knee 60 and ankle pads/braces 65, socks 70, and the like which can provide a localized treatment on the body for an extended amount of time while being hidden beneath clothing.

The drug delivery device comprises a porous material 15 that can be offered in strips or as a garment to be worn underneath clothing. The device can be provided within a topical medication filled container 75, thereby enabling the reuse of the drug delivery device 10. The present invention enables an individual to continuously receive medication on the skin in order to provide treatment for an extended period of time. Further, the present invention enables the localized application of medication in hard to reach places, such as the back of an individual. This can be accomplished by the wearing of a chest/back garment 25 or providing the topical medication on a portion of the present invention, thereby providing proper placement on the body without requiring the assistance of a helping individual.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A topical drug delivery device, said device, comprising:
a sheet of material;
said sheet comprising a woven mesh material with a plurality of pores thereon, whereby said pores are adapted for impregnation with a medical solution.
wherein said medical solution comprises one or more of iodine, caffeine, hormones, pain medication, diet drugs, argentine, taurine, vitamin B, vitamin D, ginseng, and green tea.

2. The topical drug delivery device of claim 1, wherein said sheet comprises a device adapted for wearing around the body of an individual.

3. The topical drug delivery device of claim 2, wherein said sheet comprises a strip of material that is adapted for wrapping around portions of the body of an individual.

4. The topical drug delivery device of claim 2, wherein said sheet comprises a garment adapted for wearing on the body.

5. The topical drug delivery device of claim 4, wherein said garment comprises a central opening and sides thereon adapted for placement over the chest and back of an individual.

6. The topical drug delivery device of claim 4, wherein said garment is shaped to cover the foot of an individual.

7. The topical drug delivery device of claim 4, wherein said garment is shaped to cover the knee of an individual.
